(19) United States
(12) Patent Application Publication
(10) Pub. No.: US 2010/0249551 A1
(43) Pub. Date: Sep. 30, 2010

(54) System And Method For Generating Corrective Actions Correlated To Medical Sensor Errors

(75) Inventor: Mark C. Miller, Longmont, CO (US)

Correspondence Address: NELLCOR PURITAN BENNETT LLC;ATTN: IP LEGAL, 6135 Gunbarrel Avenue, Boulder, CO, 80301, US

(73) Assignee: Nelicor Puritan Bennett LLC, Boulder, CO (US)

(21) Appl. No.: 12/415,520

(22) Filed: Mar. 31, 2009

(51) Int. Cl. A61B 5/1455

(52) U.S. Cl. 600/323

(57) ABSTRACT

A system and method for determining physiological parameters of a patient as well as errors based on light transmitted through the patient. Based on the received light, a most likely type of error may be determined, as well as one or more most likely actions to be undertaken to correct the error. Both the error and the corrective actions to be undertaken may be displayed.

### BACKGROUND

The present disclosure relates generally to medical devices and, more particularly, to determination of errors and generation of potential corrective actions for the errors.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed and/or scattered. More specifically, the light passed through the tissue is typically selected to be of one or more wavelengths that may be absorbed and/or scattered by the blood in an amount correlative to the amount of the blood constituent present in the blood. The amount of light absorbed and/or scattered may then be used to estimate the amount of blood constituent in the tissue using various algorithms.

Several optical conditions not indicative of physiologic conditions of a patient may be detected by pulse oximeters. Furthermore, a general list of errors and/or a general list of solutions may be presented to a user of the pulse oximeter when these non-physiological conditions are detected. However, because the presented solutions are general, application of the presented solutions may not aid in correcting the non-physiological conditions detected.

### BRIEF DESCRIPTION OF THE DRAWINGS

Advantages of the disclosure may become apparent upon reading the following detailed description and upon reference to the drawings in which:

FIG. 1 illustrates a perspective view of a pulse oximeter in accordance with an embodiment;

FIG. 2 illustrates a simplified block diagram of a pulse oximeter in FIG. 1, according to an embodiment;

FIG. 3A illustrates an example of a screen shot on the display of the monitor in FIG. 1, according to an embodiment;

FIG. 3B illustrates an example of another screen shot on the display of the monitor in FIG. 1, according to an embodiment;

FIG. 4 illustrates a flow chart of the operation of a pulse oximeter in FIG. 1, according to an embodiment;

FIG. 5 illustrates a flow chart in connection with an example of a signal state determination for a pulse oximeter in FIG. 1 utilizing a look-up table, according to an embodiment; and

FIG. 6 illustrates a flow chart in connection with an example of a signal state determination for a pulse oximeter in FIG. 1 utilizing a neural network, according to an embodiment.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A system and method for determination of sensor errors in a pulse oximeter is provided herein. The sensor errors may be determined in a probabilistic manner, such that a most likely reason for the error may be determined. Furthermore, one or more most likely corrective actions may be determined based on the type of error determined. Both the type of error, as well as the one or more corrective actions, may be displayed on a monitor of the pulse oximeter, thus allowing a user to view a tailored set of corrective actions, rather than, for example, being presented with a “laundry list” of all potential corrective actions

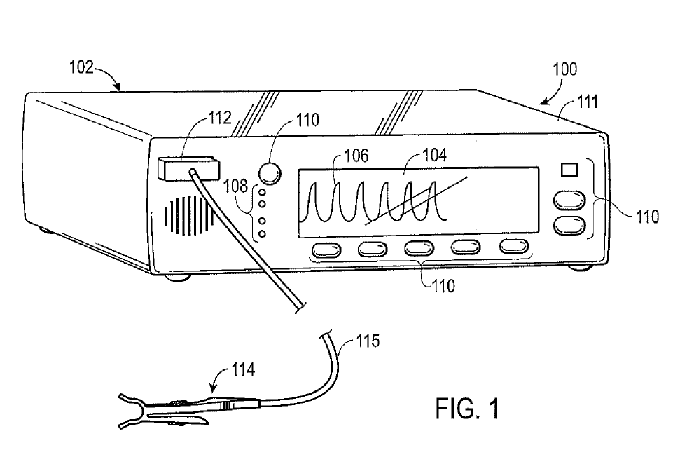

Turning to FIG. 1, a perspective view of a medical device is illustrated in accordance with an embodiment. The medical device may be a pulse oximeter 100. The pulse oximeter 100 may include a monitor 102, such as those available from Nellcor Puritan Bennett LLC. The monitor 102 may display calculated parameters on a display 104. As illustrated in FIG. 1, the display 104 may be integrated into the monitor 102. However, the monitor 102 may provide data via a port to a display (not shown) that is not integrated with the monitor 102. The display 104 may display computed physiological data including, for example, an oxygen saturation percentage, a pulse rate, and/or a plethysmographic waveform 106. As is known in the art, the oxygen saturation percentage may be a functional arterial hemoglobin oxygen saturation measurement in units of percentage SpO₂, while the pulse rate may indicate a patient's pulse rate in beats per minute. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option. The monitor 102 may also include a casing 111. The casing 111 may aid in the protection of the internal elements of the monitor 102 from damage.

The monitor 102 may further include a sensor port 112. The sensor port 112 may allow for connection to an external sensor 114, via a cable 115 which connects to the sensor port 112. The sensor 114 may be of a disposable or a non-disposable type. Furthermore, the sensor 114 may obtain readings from a patient, which can be used by the monitor to calculate certain physiological characteristics such as the blood-oxygen saturation of hemoglobin in arterial blood, the volume of individual blood pulsations supplying the tissue, and/or the rate of blood pulsations corresponding to each heartbeat of a patient.

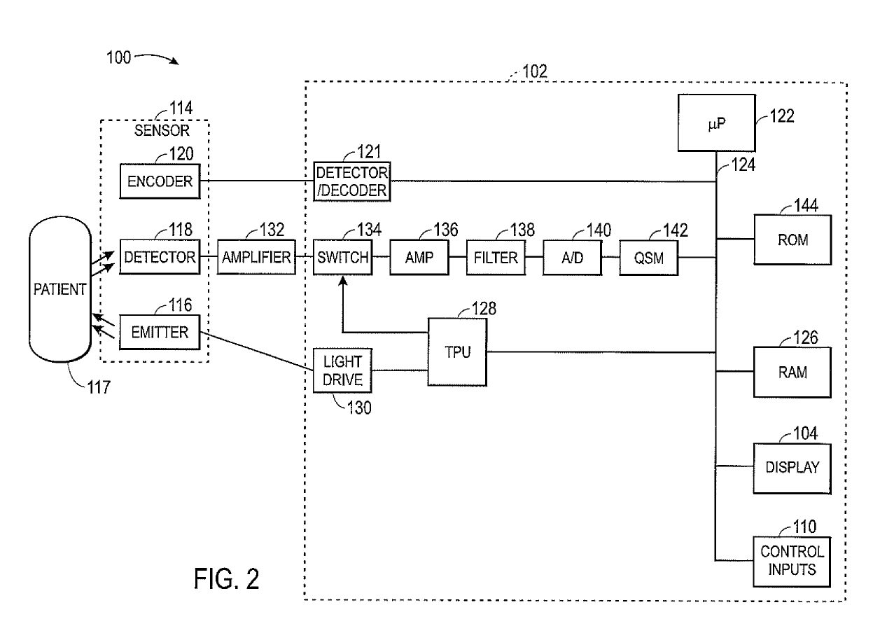

Turning to FIG. 2, a simplified block diagram of a pulse oximeter 100 is illustrated in accordance with an embodiment. The sensor 114 may contain a light source, i.e. an emitter 116, a detector 118, and an encoder 120. The emitter 116 may have one or more light sources that transmit electromagnetic radiation, i.e., light, into the tissue of a patient 117. For example, the emitter 116 may include a plurality of LEDs operating at discrete wavelengths, such as in the red and infrared portions of the electromagnetic radiation spectrum. Alternatively, the emitter 116 may be a broad spectrum emitter, or it may include wavelengths for measuring water fractions, hematocrit, or other physiologic parameters. As set forth above, light may be transmitted from the emitter 116, and may pass into a blood perfused tissue of a patient 117 whereby it may be scattered and/or absorbed, and then detected by the detector 118. The detector 118 may be a photoelectric detector which may detect the scattered and/or absorbed light from the patient 117. Based on the detected light, the detector 118 may generate an electrical signal, e.g., current, at a level corresponding to the detected light. The sensor 114 may direct the electrical signal to the monitor 102 for processing and calculation of physiological parameters.

Additionally, the sensor 114 may include an encoder 120, which may be capable of providing signals indicative of the wavelength(s) of the emitter 116 to allow the oximeter 100 to select appropriate calibration coefficients for calculating oxygen saturation of the patient. The encoder 120 may be a memory device, such as an EPROM, that stores wavelength information and/or the corresponding coefficients. The encoder 120 may be communicatively coupled to the monitor 102 in order to communicate wavelength information to the decoder 121. The decoder 121 may receive and decode the wavelength information from the encoder 120. Once decoded, the information may be transmitted to the processor 122 for utilization in calculation of the physiological parameters of the patient 117.

Accordingly, the sensor 114 may be connected to a pulse oximetry monitor 102. The monitor 102 may include a microprocessor 122 coupled to an internal bus 124. Also connected to the bus may be a RAM memory 126 and a display 104. A time processing unit (TPU) 128 may provide timing control signals to light drive circuitry 130, which controls when the emitter 116 is activated, and if multiple light sources are used, the multiplexed timing for the different light sources, TPU 128 may also control the gating-in of signals from detector 118 through an amplifier 132 and a switching circuit 134. These signals are sampled at the proper time, depending at least in part upon which of multiple light sources is activated, if multiple light sources are used. The received signal from the detector 118 may be passed through an amplifier 136, a low pass filter 138, and an analog-to-digital converter 140 for amplifying, filtering, and digitizing the electrical signals the from the sensor 114. The digital data may then be stored in a queued serial module (QSM) 142, for later downloading to RAM 126 as QSM 142 fills up. In an embodiment, there may be multiple parallel paths of separate amplifier, filter, and AID converters for multiple light wavelengths or spectra received.

In an embodiment, based at least in part upon the received signals corresponding to the light received by detector 118, microprocessor 122 may calculate the oxygen saturation using various algorithms. These algorithms may require coefficients, which may be empirically determined, and may correspond to the wavelengths of light used. The algorithms may be stored in a ROM 144 and accessed and operated according to microprocessor 122 instructions.

On occasion, the monitor 102 may receive values from the sensor 114 that are not indicative of the physiological parameters of a patient 117, but rather are indicative of non-physiologic optical conditions. For example, these non-physiologic optical conditions may be representative of a mispositioned or a removed sensor 114. Pulses that are either too weak, too strong, contain too much or too little infrared light, pulses that contain the presence of a waveform artifact, or pulses that include a high signal-to-noise ratio may all indicate problems with positioning of the sensor 114. Additionally, dysfunctional hemoglobin, arterial dyes, low perfusion, dark pigment, and/or externally applied coloring agents, such as nail polish, dye, or pigmented cream, may interfere with the ability of the pulse oximeter 100 to detect and display measurements. When any of these non-physiologic optical conditions are detected, the monitor 102 may, for example, display both the detected conditions, as well as potential solutions to correct the detected conditions.

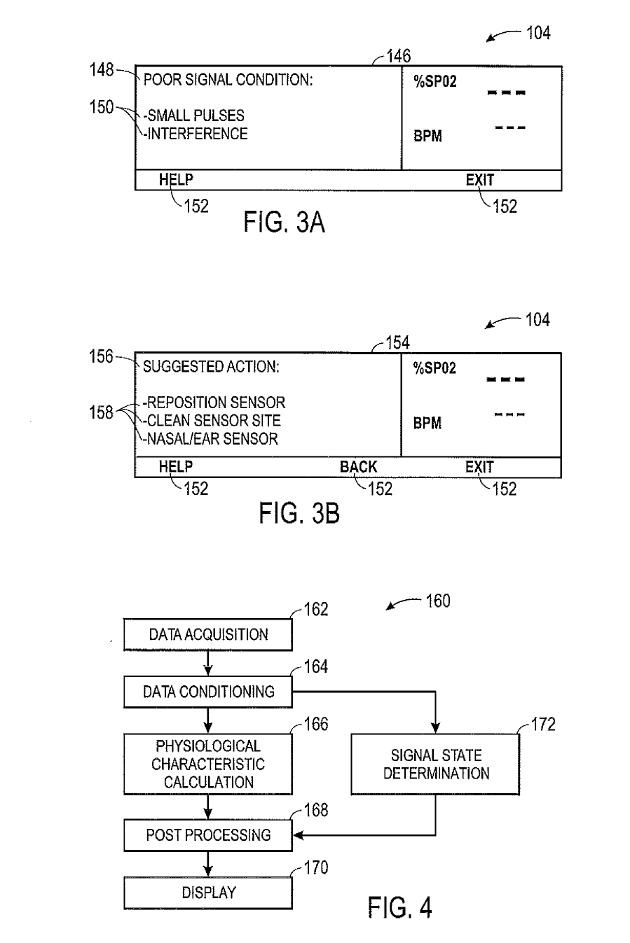

FIG. 3A illustrates an example of a sensor error message screen 146 that may be generated on the display 104 of the monitor 102 when non-physiologic optical conditions have been received and detected. The sensor error message screen 146 may include a poor signal condition indicator 148 as well as one or more sensor condition messages 150. The sensor condition messages 150 may include, for example, indicators directed to small pulses received by the detector, interference signals received at the detector 118 in combination with the desired optical signals, weak signals received at the detector, excess infrared light received at the detector, and/or invalid amplitude of the pulse signal.

Furthermore, in one embodiment, each of the sensor condition messages 150 may be generated and displayed on the display 104 based upon the probability that the received signals from the detector 118 likely are associated with the typical type of sensor error to be indicated by the sensor condition messages 150. For example, if the signals received at the detector 118 contain more infrared light than typically should be present during operation of the pulse oximeter 100, a sensor condition message 150 corresponding to excess infrared light may be displayed on the display 104. Similarly, if the signals received at the detector 118 contain a weaker signal than typically should be present during operation of the pulse oximeter 100, a sensor condition message 150 corresponding to a weak signal may be displayed on the display 104. The determination of which sensor condition messages 150 are displayed will be discussed further below.

FIG. 3A also displays one or more interface messages 152. For example, the interface messages 152 may display the term “Help” and/or “Back”. These interface messages 152 may be positioned above control inputs 110 of the monitor 102 such that activation of the respective control input 110 associated with a given interface message 152 will cause the display 104 to show information associated with the selection. For example, selection of a control input 110 associated with, i.e. beneath, the term “Exit” may cause the display 104 to display a main screen for the monitor 102. Alternatively, selection of a control input 110 associated with the term “Help” may cause the display 104 to display a suggested action screen 154, as illustrated in FIG. 3B.

FIG. 3B illustrates a suggested action screen 154 that may display a suggested action indicator 156 as well as one or more corrective action messages 158. The corrective action messages 158 may list one or more suggestions for user action that may eliminate the non-physiologic optical conditions that have caused the sensor error message 146 of FIG. 3A to be displayed. Examples of the conditions to be performed may include, for example, placing the sensor 114 on an alternate site, covering the sensor site (for example to reduce ambient light affecting the sensor 114), checking to see if the sensor 114 is for use with the nose or ear of the patient 117 (i.e., is the sensor 114 correctly placed on the nose or ear of the patient 117), determining if the sensor 114 is of a type to be used with the monitor 102 (i.e. compatible), determining if the cable 115 of the sensor 114 is secure, checking to see if the sensor 114 is for use with the ear or forehead of the patient 117 (i.e., is the sensor 114 correctly placed on the ear or forehead of the patient 117), checking if a headband is being utilized with the sensor 114, warming the site of the patient 117 before reapplying the sensor 114, checking the bandage assembly of the sensor 114, determining if nail polish is present on a patient using a finger type sensor 114, determining if the sensor 114 was applied too tightly, determining if the sensor 114 should be repositioned, determining if an interference source is proximate to the sensor 114 and removing the interference source, and/or cleaning the sensor site of the patient 117.

Furthermore, as may be seen in FIG. 3B, the display 104 may also display one or more one or more interface messages 152. For example, the interface messages 152 may display the terms “Next”, “Back”, and/or “Exit” positioned above control inputs 110 of the monitor 102 such that activation of the respective control input 110 associated with a given interface message 152 will cause the display 104 to show information associated with the selection For example, selection of a control input 110 associated with, i.e. beneath, the term “Next” may cause the display 104 to display additional corrective action messages 158. Alternatively, selection of a control input 110 associated with the term “Back” may cause the display 104 to display the sensor error message screen 146, as illustrated in FIG. 3A. Finally, selection of a control input 110 associated with the term “Exit” may cause the display 104 to display a main screen for the monitor 102.

As will be additionally discussed below, the order in which the one or more corrective action messages 158 are displayed may be determined based on their probability of correcting the detected error associated with a sensor condition message 150. That is, the corrective action message 158 most likely to correct the error that led to the generation of a sensor condition message 150 may be listed first out of all displayed corrective action messages 158. This first corrective action message 158 may be followed by a second, and subsequent corrective action messages 158 that may be next most likely to cure the error that led to the generation of a sensor condition message 150.

Turning to FIG. 4, a flow chart of a technique 160 for operation of the pulse oximeter 100 is illustrated, according to an embodiment. Technique 160 may be executed, for example, by the processor 122 of FIG. 2, by an external computer system (not illustrated) that may be coupled to the monitor 102, or, alternatively, executed in hardware/firmware of an ASIC (not shown) of the monitor 102. In this embodiment, the technique 160 begins by acquiring a signal, as indicated by block 162. In various embodiments, data acquisition may occur as the detector 118 receives the electromagnetic radiation from emitters 116 and generates a photoelectric current as data. Subsequently, data conditioning may occur, as indicated by block 164. The data conditioning of block 164 may include, for example, the analog-to-digital conversion of the data, performing the natural logarithm of digitized waveforms of the data, filtering the resulting data, e.g. bandpass filtering the data with an infinite impulse response filter (IIR) having a high pass cutoff at 0.5 Hz and a low pass roll off from 10 to 20 Hz, and/or normalizing the data, e.g., down-weighting large pulse amplitudes so that each pulse has roughly the same average amplitude, thus de-emphasizing outlying data values.

Following the conditioning of the data in block 164, physiological characteristics of the patient may be calculated based on the conditioned data, as indicated by block 166. For example, the conditioned, i.e., filtered and normalized, data may be utilized for calculation of the pulse rate and/or oxygen saturation of a patient 117 in block 166. The values calculated in block 166 may undergo post processing in block 168. The post processing step 168 may, for example, determine the reliability of the calculated values as well as whether and how the values should be displayed in step 170.

The conditioned data is also provided for a signal state determination, in block 172. The signal state determination of block 172 may determine if the received data falls outside of certain ranges of acceptable data tolerances. If so, a probabilistic determination may be made as to both the causes and potential cures for the faulty data. The results of the signal state determination 172 may be provided as another input for the post processing of step 168, so that an appropriate decision may be made as to whether and how to display the current values reported by the detector 118.

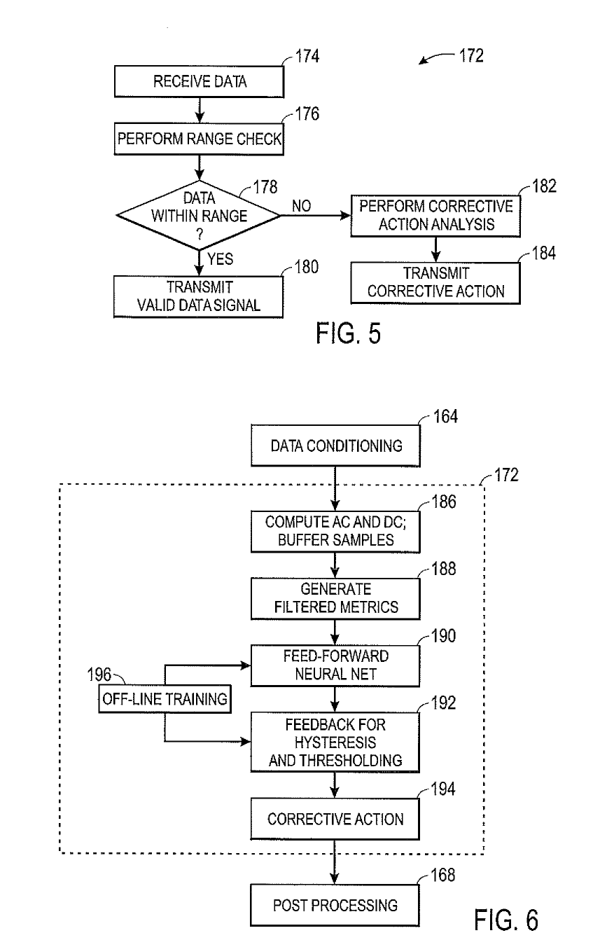

FIG. 5 illustrates the signal state determination step 172 in greater detail. The signal state determination step 172 may begin with receiving the conditioned data, described above, in step 174. A range check may be performed on the data in step 176, for example, by the microprocessor 122. The range check 176 may include determination of whether the received data falls outside of determined boundaries. For example, the signal-to-noise ratio of the data may be determined, which may then be checked against a range of typical values in step 176. Additionally, more than one data value may be checked against various ranges, either sequentially, or simultaneously. The range of typical values used in the comparison may, for example, be stored in a look-up table in the RAM 126 of the monitor 102 and these ranges may be stored prior to operation of the pulse oximeter 100.

In step 178 a determination of whether the data is within the acceptable range is made. If the data value falls within the accepted range in step 178, a valid data signal is transmitted in step 180 as an input to be utilized in the post processing step 168 described above. If, however, the data falls outside the accepted range of step 178, a corrective action analysis 182 may be performed.

The corrective action analysis 182 may be performed, for example, using a probabilistic state transition scheme. This probabilistic state transition scheme may include mapping the conditioned data against trained data, whereby the trained data may include actual data results preprogrammed into the pulse oximeter. That is, the pulse oximeter 100 may include a set of trained data that corresponds to input data values and corrective actions that corrected the one or more causes of the input data value. For example, a first infrared input data value may be stored as corresponding to a sensor having fallen off a patient 117, while a second infrared input data value may correspond to an ear sensor having been placed on the nose of a patient 117.

Accordingly, the corrective action analysis 182 may compare the conditioned data against the trained data and may determine, for example, if the conditioned data matches the first or second infrared input data values. If either infrared input data value matches the conditioned data, the appropriate response may be transmitted as a corrective action 184. For example, if it is determined that the conditioned data matches the second infrared input data, then a response corresponding to a corrective action of checking to see if the sensor is an ear sensor placed in the nose of a patient 117 may be transmitted, as seen in block 184. Furthermore, if the conditioned data matches more than one input data value, then a response corresponding to a corrective action of all matching input data values may be transmitted, as seen in block 184. Similarly, if the conditioned data matches no input data values, then a response corresponding to a corrective action for the input data value closest to the conditioned data may be transmitted, as illustrated in block 184, as the most likely action to correct generation of the error. Furthermore, it should be noted that the input data values may be updated through the use of historical data. That is, if a certain corrective action performed by a user corrects a given error in the conditioned data, then it may be added as input data and tied to that corresponding corrective action for future use in performing a corrective action analysis 182. In one embodiment, the added input data may be added via inputs 110.

Performing a corrective action analysis 182 may, in an embodiment, utilize a neural network. In accordance with an embodiment, metrics may be used in a neural network to determine the probability of a given error type and the corresponding corrective action based on received signals from the detector 114. For example, one error type may be whether the sensor 114, for example, is in contact with tissue of the patient 117. The corrective action for this error type may be to adjust the placement of the sensor 114. The neural network may be executed, for example, by the processor 122 of FIG. 2, by an external computer system (not illustrated) that may be coupled to the monitor 102, or, alternatively, executed in hardware/firmware of an ASIC (not shown) of the monitor 102.

Neural networks may generally be represented symbolically as an interconnected network of nodes arranged in a specific topology or configuration. Links between nodes represent dependencies between nodes and have weights associated with each link representing the strengths of the dependencies. Artificial neural networks are often used to represent or process nonlinear functions applied to large data sets, Artificial neural network engines can be implemented in software, hardware (using parallel processing architectures) or a combination of both and neural networks may be well-suited for detecting trends or patterns in data.

Artificial neural networks are represented symbolically as an interconnected network of nodes arranged in a specific topology or configuration. Links between nodes represent dependencies between nodes and have weights associated with each link representing the strengths of the dependencies. Artificial neural networks typically have an input layer, hidden or processing layers, and an output layer. The links between nodes are adjusted for specific tasks by training of the network, which involves exposing the network to representative data sets to be processed. Output from the network may be compared to desired results and corresponding adjustments may be made to reduce any discrepancies between the desired output and the actual output. The metrics described herein include inputs to the neural network and quantify aspects of the behavior of data retrieved over a period of several seconds.

A feedback layer may provide threshold comparison information for determining the probability of a particular condition of the sensor, i.e., whether it is in contact with arterialized tissue or not, and may have built in hysteresis. The coefficients for both the neural network and feedback layer may be determined by off-line training algorithms which are responsible for finding and optimizing the relationships between the inputs and the outputs, described in more detail below.

The discussion below will focus on utilization of conditioned data signals to determine whether the sensor 114 is in contact with arterialized tissue from the feedback layer as well as delivery of one of a plurality of sensor state indications to a processing subsystem, such as the microprocessor 122, as well as corrective actions corresponding to the sensor state indications. However, it should be noted that the contact of the sensor 114 with a patient 117 is merely one example of a situation to be ascertained from the conditioned data and that other situations may equally be found in a manner substantially equivalent to that discussed below.

According to an embodiment, FIG. 6 illustrates the signal state determination block 172 in connection with a neural network in greater detail. As illustrated in FIG. 6, after the data has been conditioned in block 164, alternating current (AC) and direct current (DC) elements of the data are computed and signal samples may be buffered, as indicated in step 186. The AC and DC components of, for example, the red and infrared waveforms are used as inputs to metric computation in which the individual metrics are computed, clipped, and averaged, yielding, for example, filtered metrics in step 188. Examples of these filtered metrics are described below.

One filtered metric may correspond to an average infrared (IR) AC amplitude. This metric is sensitive to rapid changes in light absorption. The IR channel may used because the IR light level is less affected by large oxygen saturation changes than the red light level is. Accordingly, because light level can change drastically when the sensor comes off, this metric may provide a good indication of that occurrence. Other filtered metrics may include the relative variability of the IR AC amplitude, a metric based on the degree to which the IR and Red AC-coupled waveforms are correlated (which may change based on motion artifacts), a metric based on the variability of the IR direct current (DC) light level, a metric indicating the bias or slope of the IR DC light level, and/or a metric representative of the pulse shape.

At every sample, the signal state is determined 172 by analyzing several metrics computed from the IR and Red analog-to-digital converted (AD C), normalized, and derivative filtered values, in conjunction with the system gains and flags indicating the validity of these values. A Sensor Valid flag, for example, may indicate whether the sensor 114 is connected to the monitor 102. Various metrics may be computed using the combined waveforms as discussed in detail above, as indicated at block 190. The metrics may then be provided to a feed-forward neural network, as indicated at block 192. That is, the neural net receives the input metrics defined above and determines whether what type of error has occurred to generate the specific conditioned data, as well as what corrective action to take based the type of error that has occurred, i.e., the neural network may determine the probability of the state of the sensor 112 according to the metrics which have been computed.

Thus, the filtered metrics may be transmitted for use by a feed-forward neural net in step 190, to determine whether the sensor 114 is properly connected to the patient 117 and to generate a value representing the probability thereof, as well as the one or more most likely corrective actions corresponding to the determination of the proper connection of the sensor 114. The probability may be presented as feedback for hysteresis and thresholding, as indicated at block 192, which may determine a corrective action corresponding to the state of the sensor 114. This corrective action, in step 194, may be transmitted for post processing in step 168 for eventual display as an indication of both the error and corrective action to be undertaken. Offline training of the neural network and feedback may be provided as indicated at block 196. Alternatively, a no corrective action signal may be transmitted in step 194 if no errors in the conditioned data are determined to exist.

As illustrated in block 196, the neural net may receive offline training. This training may yield, for example, a feed-forward network with a ten-node hidden layer and a single-node output layer where all nodes are fully connected, and have associated bias inputs. Thus, all nodes in a layer may receive the same inputs, although those inputs may have different weights and biases. The inputs to the hidden layer may be the filtered input metrics described above. The inputs to the output layer may be the outputs of the ten hidden nodes in the hidden layer. The neural net's training goal is to accurately output the probability (between 0 and 1) that the sensor 112 is in a given condition, given the values of the neural net's input metrics. To do this, the neural net's hidden nodes may collectively allow the neural net to map the “boundary” of the region of this input space in which resultant data are believed to lie.

Thus, training of the neural net may include injecting a given test sequence, weighting the signal metrics, and determining the outcome. This procedure may be repeated to generate a matrix with all outcomes that may then be compared to and input conditioned data. Furthermore, a number of neural-net training techniques, such as the Levenberg-Marquardt back-propagation method, are known to those skilled in the art of signal processing. The neural network may be trained according to an algorithm that is responsible for finding and optimizing the relationships between the neural network's inputs (the metrics) and the output. For example, the trained neural network may be implemented as an array of dot products and functions. Additionally the neural network may be retrained by providing updated coefficients. The neural network may be trained on a large database containing data representative of different sensor states, whereby the data may be classified as indicating the sensor state and, therefore, may be used to set the thresholds for a probability determination of sensor states by the neural network based on the input metrics.

Regardless of the method utilized to determine the signal state of block 172, based on the corrective actions received in the post processing step 164, error messages corresponding to data errors in the received data from the sensor may be displayed on the display 104, as previously discussed with respect to FIG. 3A. Furthermore, a lost of one or more of the most likely corrective actions to be undertaken by a user to correct the data errors may be displayed on the display 104, as previously discussed with respect to FIG. 3B. In this manner, a user may be provided with a tailored set of corrective actions to correct data recovery errors associated with a sensor 114, rather than, for example, being presented with a “laundry list” of potential corrective actions. Furthermore, it should be noted that the techniques described in FIGS. 4-6 may be implemented via software, hardware, or some combination thereof. For example, instructions corresponding to the steps to be performed in each of FIGS. 4-6 may be stored on one or more tangible machine readable mediums such as the RAM 126, and may be executed by, for example, the microprocessor 122.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. A pulse oximeter comprising:
a processor capable of determining if a signal received by the pulse oximeter corresponds generally to a detection error, and probabilistically determining a corrective action based at least in part upon the detection error.

2. The pulse oximeter, as set forth in claim 1, comprising a display capable of displaying an indication of the corrective action.

3. The pulse oximeter, as set forth in claim 1, wherein the processor comprises a neural network capable of determining if the signal generally corresponds to the detection error.

4. The pulse oximeter, as set forth in claim 1, wherein the processor comprises a neural network capable of determining a corrective action based at least in part upon the detection error.

5. The pulse oximeter, as set forth in claim 1, comprising memory capable of storing range data corresponding generally to the signal for use in probabilistically determining a correspondence between the signal and the detection error.

6. The pulse oximeter, as set forth in claim 1, wherein the processor is capable of computing a physiological parameter based at least in part upon the received signal.

7. A non-invasive medical device, comprising:
a sensor comprising:
a light emitting diode capable of transmitting electromagnetic radiation; and
a photodetector capable of detecting the electromagnetic radiation and generating electrical signals based at least in part upon the detected electromagnetic radiation; and
a monitor coupled to the sensor, wherein the monitor is capable of:
transforming the electronic signals into conditioned data based at least in part upon analog-to-digital conversion of the electronic signals and filtering of the electronic signals;
determining the signal state of the conditioned data, wherein the signal state of the conditioned data is based at least in part upon a condition of the sensor;
determining a most likely corrective action, wherein the corrective action corresponds to an alteration of the condition of the sensor.

8. The non-invasive medical device of claim 7, wherein the monitor is capable of determining a second most likely corrective action.

9. The non-invasive medical device of claim 8, wherein the monitor comprises a display capable of displaying an indication of the most likely corrective action and the second most likely corrective action.

10. The non-invasive medical device of claim 7, wherein the monitor comprises a neural network capable of determining a most likely condition of the sensor based at least in part upon a matrix of outcomes.

11. The non-invasive medical device of claim 7, wherein the monitor comprises a memory, wherein the memory is capable of storing range data corresponding to the conditioned data and corrective actions based at least in part upon the range data.

12. The non-invasive medical device of claim 11, wherein the monitor comprises inputs, wherein the inputs are capable of updating the range data and the corrective actions.

13. A method comprising:
receiving a signal in a pulse oximeter;
determining if the signal corresponds to an error; and
probabilistically determining a corrective action based at least in part upon the error.

14. The method of claim 13, comprising displaying an indication of the error.

15. The method of claim 13, comprising displaying an indication of the corrective action.

16. The method of claim 13, wherein determining if the signal corresponds to an error comprises transmitting the signal to a neural network capable of determining the error based at least in part on a matrix of outcomes.

17. The method of claim 16, wherein probabilistically determining a corrective action comprises determining the corrective action from a set of corrective actions wherein each corrective action corresponds to each outcome in the matrix of outcomes.

18. The method of claim 17, comprising updating the matrix of outcomes with manually inputted error information.

19. The method of claim 13, wherein determining if the signal corresponds to an error comprises comparing the signal with range data corresponding to expected values for the signal.

20. The method of claim 19, wherein probabilistically determining a corrective action corresponding to the error comprises determining the corrective action from a set of corrective actions corresponding to the range data.
